(19) United States
(12) Patent Application Publication
(10) Pub. No.: US 2022/0280789 A1
(43) Pub. Date: Sep. 8, 2022

(54) AUTOMATIC ECAP ELECTRODE SELECTION AND MAINTENANCE

(71) Applicant: Boston Scientific Neuromodulation Corporation, Valencia, CA (US)

(72) Inventors: John Rivera, Oxnard, CA (US); David Michael Wagenbach, Simi Valley, CA (US); Philip Leonard Weiss, Sherman Oaks, CA (US)

(21) Appl. No.: 17/685,083

(22) Filed: Mar. 2, 2022

Related U.S. Application Data
(60) Provisional application No. 63/156,699, filed on Mar. 4, 2021.

(51) Int. Cl. A61N 1/36; A61N 1/05; A61N 1/372

(52) U.S. Cl. CPC A61N 1/3614; A61N 1/36192; A61N 1/0551; A61N 1/36132; A61N 1/37247

(57) ABSTRACT

A system may include an implantable device and a controller. The implantable device may include sensing-capable electrodes. The controller may be configured to receive a trigger signal indicative of a trigger to evaluate sensing capabilities of the sensing capable electrodes, respond to the received trigger signal by evaluating the sensing capabilities of the sensing-capable electrodes to assess or reassess which of the sensing-capable electrodes are available to be activated for sensing ECAPs, activate at least one of the sensing-capable electrodes that are available to be activated based on the evaluating the sensing capabilities, and sense the ECAPs using the activated ones of the sensing-capable electrodes.

### CLAIM OF PRIORITY

This application claims the benefit of U.S. Provisional Application No. 63/156,699, filed on Mar. 4, 2021, which is hereby incorporated by reference in its entirety.

### TECHNICAL FIELD

This document relates generally to medical systems, and more particularly, but not by way of limitation, to systems, devices, and methods for sensing nerve activity.

### BACKGROUND

Implantable devices may be configured to sense nerve activity such as evoked compound action potentials (ECAPs). The neural sensor may be its own device, or may be part of a therapy-delivery device. The delivered therapy may include electrical therapy or drug therapy, for example. By way of example, the implantable devices may be neuromodulators that are also capable of delivering neuromodulation therapy. An example may also include cardiac stimulators that monitor nerve activity.

Neuromodulation, also referred to as neurostimulation, has been proposed as a therapy for a number of conditions. Examples of neuromodulation include Spinal Cord Stimulation (SCS), Deep Brain Stimulation (DBS), Peripheral Nerve Stimulation (PNS), and Functional Electrical Stimulation (FES). Implantable neuromodulation systems have been applied to deliver such a therapy. An implantable neuromodulation system may include an implantable neuromodulator, also referred to as an implantable wave generator or an implantable pulse generator (IPG), and one or more implantable leads each including one or more electrodes. The implantable neuromodulator delivers neuromodulation energy through one or more electrodes placed on or near a target site in the nervous system. An external programming device may be used to program the implantable neuromodulator with modulation parameters controlling the delivery of the neuromodulation energy. The neuromodulation energy may be delivered using an electrical modulation waveform, which may be defined by a plurality of modulation parameters. For example, electrical modulation waveform may be an electrical pulsed waveform. Other parameters that may be controlled or varied include the electrodes within the electrode array that are activated, the amplitude, pulse width, and rate (or frequency) of the electrical pulses provided to individual ones of the activated electrodes.

### SUMMARY

An example (e.g. “Example 1”) of a system may include an implantable device and a controller. The implantable device may include sensing-capable electrodes. The controller may be configured to receive a trigger signal indicative of a trigger to evaluate sensing capabilities of the sensing capable electrodes, respond to the received trigger signal by evaluating the sensing capabilities of the sensing-capable electrodes to assess or reassess which of the sensing-capable electrodes are available to be activated for sensing ECAPs, activate at least one of the sensing-capable electrodes that are available to be activated based on the evaluating the sensing capabilities, and sense the ECAPs using the activated ones of the sensing-capable electrodes.

In Example 2, the subject matter of Example 1 may optionally be configured such that the controller is configured to respond to the received trigger by measuring an impedance corresponding to each of at least one of the sensing-capable electrodes, and evaluating the measured impedance against threshold values to assess or reassess which of the sensing-capable electrodes are available to be activated for sensing ECAPs.

In Example 3, the subject matter of Example 2 may optionally be configured such that the controller is configured to remove at least one electrode, based on the evaluating the measured impedance against the threshold values, from the sensing-capable electrodes that are available to be activated.

In Example 4, the subject matter of any one or any combination of Examples 2-3 may optionally be configured such that the controller is configured to add at least one electrode, based on the evaluating the measured impedance against the threshold values, to the sensing-capable electrodes that are available to be activated.

In Example 5, the subject matter of any one or any combination of Examples 1-4 may optionally be configured to further include a user interface for receiving a user-input, wherein the trigger signal is indicative of the user input.

In Example 6, the subject matter of any one or any combination of Examples 1-5 may optionally be configured to further include a memory configured to be programmed with a schedule, wherein the trigger signal is provided in accordance with the programmed schedule.

In Example 7, the subject matter of any one or any combination of Examples 1-6 may optionally be configured to further include at least one sensor for sensing at least one physiological parameter and to provide the trigger signal based on the sensed at least one physiological parameter.

In Example 8, the subject matter of any one or any combination of Examples 1-7 may optionally be configured to further include an ECAP analyzer configured for monitoring the sensed ECAPs and to provide the trigger signal based on the monitored sensed ECAPs.

In Example 9, the subject matter of any one or any combination of Examples 1-8 may optionally be configured such that the controller is configured to reconfigure sensing configurations to create a different differential pair when at least one electrode in an existing differential pair is to be removed, or automatically replace a single electrode when another single electrode is removed.

In Example 10, the subject matter of any one or any combination of Examples 1-9 may optionally be configured such that the controller is configured to provide report data used to generate a sensing map report that identifies at least one electrode added to the sensing-capable electrodes that are available to be activated for sensing ECAPs or that identifies at least one electrode removed from the sensing-capable electrodes that are available to be activated for sensing ECAPs.

In Example 11, the subject matter of any one or any combination of Examples 1-10 may optionally be configured such that the controller is configured to provide report data used to generate a sensing map report that identifies the sensing-capable electrodes that are available to be activated for sensing ECAPs.

In Example 12, the subject matter of any one or any combination of Examples 1-11 may optionally be configured such that the evaluating the sensing capabilities of the sensing-capable electrodes includes measuring impedance for individual ones of the sensing capable electrodes.

In Example 13, the subject matter of any one or any combination of Examples 1-12 may optionally be configured such that the evaluating the sensing capabilities includes comparing a measured impedance corresponding to an electrode to threshold values for the electrode.

In Example 14, the subject matter of Example 13 may optionally be configured such that the evaluating the sensing capabilities further includes recording a violation when the measured impedance is outside of the threshold values, determining that recorded violations break a rule for allowable violations, and updating the sensing-capable electrodes that are available to be activated for sensing ECAPs.

In Example 15, the subject matter of Example 14 may optionally be configured such that the evaluating the sensing capabilities further includes updating a sensing electrode distribution record.

Example 16 includes subject matter (such as a method, means for performing acts, machine readable medium including instructions that when performed by a machine cause the machine to performs acts, or an apparatus to perform) for programming a neuromodulator to deliver neuromodulation to at least two neuromodulation sites. The subject matter may include receiving a trigger signal indicative of a trigger to evaluate sensing capabilities of the sensing capable electrodes, responding to the received trigger signal by evaluating the sensing capabilities of the sensing-capable electrodes to assess or reassess which of the sensing-capable electrodes are available to be activated for sensing ECAPs, activating at least one of the sensing-capable electrodes that are available to be activated based on the evaluating the sensing capabilities, and sensing the ECAPs using the activated ones of the sensing-capable electrodes.

In Example 17, the subject matter of Example 16 may optionally be configured such that the evaluating the sensing capabilities of the sensing-capable electrodes includes measuring an impedance corresponding to each of at least one of the sensing-capable electrodes, and evaluating the measured impedance against threshold values to assess or reassess which of the sensing-capable electrodes are available to be activated for sensing ECAPs.

In Example 18, the subject matter of any one or any combination of Examples 16-17 may optionally be configured to further comprise disabling at least one electrode, based on the evaluating the measured impedance against the threshold values, from being available to be activated.

In Example 19, the subject matter of any one or any combination of Examples 16-18 may optionally be configured to further comprise enabling at least one electrode, based on the evaluating the measured impedance against the threshold values, to be available to be activated.

In Example 20, the subject matter of any one or any combination of Examples 16-19 may optionally be configured to include receiving a user-input via a user input, wherein the trigger signal is indicative of the user input.

In Example 21, the subject matter of any one or any combination of Examples 16-20 may optionally be configured to include accessing a scheduled programmed in a memory, wherein the trigger signal is provided in accordance with the programmed schedule.

In Example 22, the subject matter of any one or any combination of Examples 16-21 may optionally be configured to include using at least one sensor to sense at least one physiological parameter and providing the trigger signal based on the sensed at least one physiological parameter.

In Example 23, the subject matter of any one or any combination of Examples 16-22 may optionally be configured to include monitoring the sensed ECAPs and providing the trigger signal based on the monitored sensed ECAPs.

In Example 24, the subject matter of any one or any combination of Examples 16-23 may optionally be configured to include reconfiguring sensing configurations to create a different differential pair when at least one electrode in an existing differential pair is to be removed.

In Example 25, the subject matter of any one or any combination of Examples 16-24 may optionally be configured to include reconfiguring sensing configurations to automatically replace a single electrode when another single electrode is removed.

In Example 26, the subject matter of any one or any combination of Examples 16-25 may optionally be configured to include generating a sensing map report that identifies at least one electrode added to the sensing-capable electrodes that are available to be activated for sensing ECAPs.

In Example 27, the subject matter of any one or any combination of Examples 16-23 may optionally be configured to include generating a sensing map report that identifies at least one electrode removed from the sensing-capable electrodes that are available to be activated for sensing ECAPs.

In Example 28, the subject matter of any one or any combination of Examples 16-27 may optionally be configured to include generating a sensing map report that identifies the sensing-capable electrodes that are available to be activated for sensing ECAPs.

In Example 29, the subject matter of any one or any combination of Examples 16-28 may optionally be configured such that the evaluating the sensing capabilities of the sensing-capable electrodes includes measuring impedance for individual ones of the sensing capable electrodes.

In Example 30, the subject matter of any one or any combination of Examples 16-29 may optionally be configured such that the evaluating the sensing capabilities includes comparing a measured impedance correspond to an electrode to threshold values for the electrode.

In Example 31, the subject matter of Example 30 may optionally be configured such that the evaluating the sensing capabilities further includes recording a violation when the measured impedance is outside of the threshold values, determining that recorded violations break a rule for allowable violations, and updating the sensing-capable electrodes that are available to be activated for sensing ECAPs.

In Example 32, the subject matter of Example 31 may optionally be configured such that the evaluating the sensing capabilities further includes updating a sensing electrode distribution record.

Example 33 includes subject matter (such as a device, apparatus, or machine) that may include a non-transitory machine-readable medium including instructions, which when executed by a machine, cause the machine to perform a method. The method may comprise receiving a trigger signal indicative of a trigger to evaluate sensing capabilities of the sensing capable electrodes, responding to the received trigger signal by evaluating the sensing capabilities of the sensing-capable electrodes to assess or reassess which of the sensing-capable electrodes are available to be activated for sensing ECAPs, activating at least one of the sensing-capable electrodes that are available to be activated based on the evaluating the sensing capabilities, and sensing the ECAPs using the activated ones of the sensing-capable electrodes.

In Example 34, the subject matter of Example 33 may optionally be configured such that the evaluating the sensing capabilities of the sensing-capable electrodes includes measuring an impedance corresponding to each of at least one of the sensing-capable electrodes, and evaluating the measured impedance against threshold values to assess or reassess which of the sensing-capable electrodes are available to be activated for sensing ECAPs.

In Example 35, the subject matter of any one or any combination of Examples 33-34 may optionally be configured to include generating a sensing map report that identifies the sensing-capable electrodes that are available to be activated for sensing ECAPs.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neuromodulation system.

FIG. 2 illustrates, by way of example, an embodiment of a stimulation device and a lead system.

FIG. 3 illustrates, by way of example, an embodiment of a programming device.

FIG. 4 illustrates, by way of example and not limitation, an embodiment of an implantable pulse generator (IPG) and percutaneous leads.

FIG. 5 illustrates an example of an implantable neuromodulation system and portions of an environment in which system may be used.

FIG. 6 provides, by way of example and not limitation, an illustration of sensing-capable electrodes for a lead, enabled (or allowed) electrodes to be active for sensing, and active ones of the electrodes for sensing.

FIG. 7 illustrates, by way of example and not limitation, a method for configuring and using sensing electrodes to sense evoked compound action potentials (ECAPs).

FIG. 8 illustrates, by way of example and not limitation, a method for evaluating sensing-capable electrodes.

FIG. 9 illustrates, by way of example and not limitation, a method for configuring and using sensing electrodes to sense evoked compound action potentials (ECAPs) where the impedance is measured using sub-threshold modulation.

FIG. 10 illustrates, by way of example and not limitation, an embodiment of reporting data provided in a form of a clinical sensing map.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

ECAPs may be sensed using implanted electrodes. However, implanted electrodes may be adversely affected by changes over time such as impedance variation, lead migration, scar tissue, and the like. The present subject matter may account for such changes over time to provide meaningful sensed data by automatically selecting and maintaining sensing electrodes using automated impedance measurements and sensing electrode configuration management. Thus, the present subject matter allows sensing to be performed with little to no need of patient intervention or corrective clinician visits, while ensuring optimal sensing results between clinical visits.

The present subject matter may provide various features using programmable hardware and associated firmware within an implantable device. These features of the implantable device may be useful for implementing various embodiments of the present subject matter. For example, the implantable device may be configurable to provide per-electrode amplitude settings (e.g., multiple independent current controlled sources for each electrode), per-electrode polarity settings (anode, cathode), and multiple channels enabling programmable pulse-width and rate parameters per-electrode group. The implantable device may be configured to provide analog measurements using analog/digital converters (ADC). An electrode-centric algorithm may be used to implement an impedance measurement voting scheme. The present subject matter may record report data, such as a matrix of per-electrode or per electrode group (differential for example) sensing, electrode identifiers, differential pair group numbers, number of violations, and average impedance measurement value for successive violations. A programmable (e.g., user-programmable or predefined) maximum number of successive violations may be used to initiate electrode redistribution, allowable electrode pairs (differential use case), impedance measurement threshold values (min-max violation values), and programmable measurement and evaluation intervals. By way of example, the system may be programmed to evaluate sensing electrodes every pulse or after a set number of pulses, every hour, every 5 hours, every day, and the like.

Firmware may iterate over all identified sensing capable electrodes and perform impedance measurements. For each electrode, impedance may be measured using a per-electrode impedance measuring current amplitude that is based (e.g. a percentage or offset) of P_{T}. The impedance measurement may be qualified against measurement threshold values, such as a minimum allowed impedance value and a maximum allowed impedance value. These values may be based on clinical-determined values for the implanted electrodes, and then saved in the implantable device. If, upon comparing the impedance measurement to the thresholds, it is determined that the impedance measurement is outside of the range, then the violation may be recorded in a log of violations. If successive violations exceed a maximum count of successive violations, then the electrode being qualified maybe disabled, and a replacement electrode may be selected from an allowable electrode list. If the electrode that is being disabled is part of the differential pair of electrodes used to sense, then the replacement electrode may be selected from an allowable differential pairing list. The sensing electrode distribution may be recorded to create a new distribution record or to update the record. The distribution record data may be viewable as a clinical sensing map via an external programmer.

The present subject matter may use information concerning electrode quality in sensing decision making algorithms. An example of such an algorithm may be modifying stimulation parameters based on the quality of the electrode. The present subject matter may create and maintain logs concerning the monitored electrode quality, and provide viewable reports regarding the sensing-capable electrodes, such as the impedance value and status (e.g., added, removed, etc.) of a given electrode.

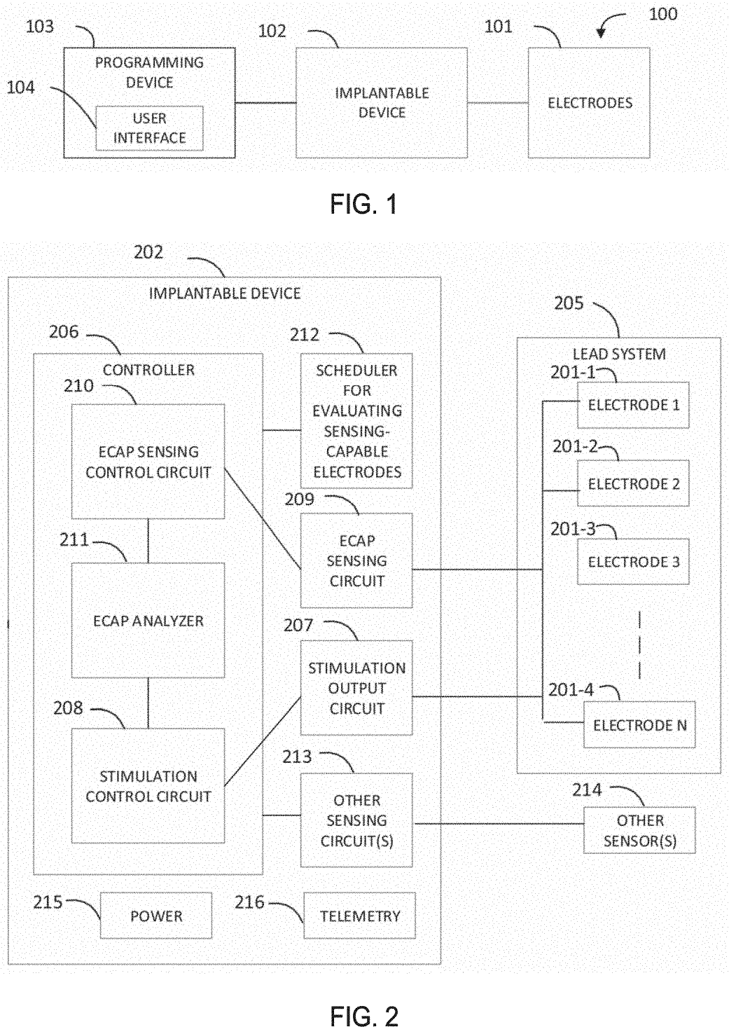

FIG. 1 illustrates, by way of example, an embodiment of a neuromodulation system. The illustrated neuromodulation system 100 includes electrodes 101, an implantable device 102, and a programming system such as a programming device 103. The programming system may include multiple devices. The electrodes 101 are configured to be placed on or near one or more neural targets in a patient. The implantable device 102 is configured to be electrically connected to electrodes 101. The implantable device 102 may be configured to sense nerve activity (e.g., ECAPs) using the electrodes 101, and may be further configured to evaluate sensing-capable electrodes as discussed within this document. The implantable device 102 may be also configured to deliver an electrical therapy such as a cardiac rhythm management therapy or a neuromodulation therapy. Therefore, by way of example and not limitation, the implantable device 102 may be a neuromodulator configured to deliver neuromodulation energy, such as in the form of electrical pulses, to the one or more neural targets though electrodes 101. The delivery of the neuromodulation may be controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver. The programming device 103 provides the user with accessibility to the user-programmable parameters. In various embodiments, the programming device 103 is configured to be communicatively coupled to modulation device via a wired or wireless link. In various embodiments, the programming device 103 includes a user interface 104 such as a graphical user interface (GUI) that allows the user to set and/or adjust a sensing configuration of the electrodes, a procedure for selecting and maintaining sensing electrodes, and/or values of the user-programmable modulation parameters.

FIG. 2 illustrates, by way of example, an embodiment of an implantable device 202 and a lead system 205, such as may be implemented in the neuromodulation system. The implantable device 202 may represent an example of the implantable device 102 in FIG. 1. The implantable device 202 may include a controller 206 for use in controlling various functions of the implantable device. The implantable device 202 may be configured to sense ECAPs and to automatically select and maintain ECAP-sensing electrodes for use in sensing the ECAPs over an extended period of time.

The implantable device 202 may include a stimulation output circuit 207 and the controller 206 may include a stimulation control circuit 208 configured for controlling the stimulation output circuit 207. The stimulation output circuit 207 may produce and deliver a neuromodulation waveform. Such waveforms may include different waveform shapes. The waveform shapes may include regular shapes (e.g., square, sinusoidal, triangular, saw tooth, and the like) or irregular shapes. The stimulation control circuit 208 may control which electrodes are used to deliver stimulation and may control the delivery of the neuromodulation waveform using the plurality of stimulation parameters, which specifies a pattern of the neuromodulation waveform. The lead system 205 may include one or more leads each configured to be electrically connected to stimulation device and a plurality of electrodes distributed in the one or more leads. In an example, the number of leads and the number of electrodes on each lead depend on, for example, the distribution of target(s) of the neuromodulation and the need for controlling the distribution of electric field at each target. In an example, the lead system includes 2 leads each having 8 electrodes. The plurality of electrodes may include electrode 201-1, electrode 201-2, electrode 201-3 . . . and electrode 201-N. The implantable device 202 may individually select electrodes to be active to provide an electrical interface between the stimulation output circuit 207 and the tissue of the patient. The neuromodulation waveform may be delivered from stimulation output circuit 207 through a set of active electrodes selected from electrodes 201-1 through 201-N.

The implantable device 202 may include an ECAP sensing circuit 209 and the controller 206 may include an ECAP sensing control circuit 210 configured for controlling the ECAP sensing circuit 209. The implantable device 202 may be configured to individually select electrodes for use to sense electrical activity in neural tissue. The ECAP sensing circuit may include amplifiers and filters for use to detect the ECAPs in the neural tissue. The ECAP sensing control circuit 210 may control the electrodes that are used to sense the ECAPs and may also be configured to perform the evaluation of the sensing-capable electrodes in the lead system 205, which will be described in more detail below.

The controller 206 may further include an ECAP analyzer 211 configured to evaluate the detected ECAPs. By way of example and not limitation, various features in the detected ECAPs may be used to control a therapy and/or monitor an efficacy of the therapy. By way of example and not limitation, the present subject matter may use characteristics of the sensed ECAPs, such as low amplitude or a significant change in detected ECAP amplitude compared to a threshold or trend, to trigger the evaluation of sensing-capable electrodes. By way of example and not limitation, the implantable device 202 may include a scheduler 212, which either may or may not form part of the controller 206, to provide a programmed schedule for triggering the evaluation of sensing-capable electrodes. By way of example and not limitation, the implantable device 202 may include other sensing circuit(s) 213 to interface with other sensor(s) 214. By way of example, these sensor(s) may be used to control a therapy and/or monitor an efficacy of the therapy. These sensor(s) may be used to trigger the evaluation of sensing-capable electrodes. For example, the sensor(s) may be used to detect significant patient activity or motion or posture changes, and the evaluation of sensing-capable electrodes may be triggered based at least in part on the detected activity, motion or posture. For sensor(s) used to monitor the efficacy of the therapy, the system may be configured to trigger the evaluation of sensing-capable electrodes when the monitored efficacy of therapy is worse than expected or is trending lower. The implantable device may include a power source 215, such as a rechargeable battery or a passive energy source configured to receive power from an external device, and may further include telemetry 216 for communicating with an external device such as the programming device 103 in FIG. 1.

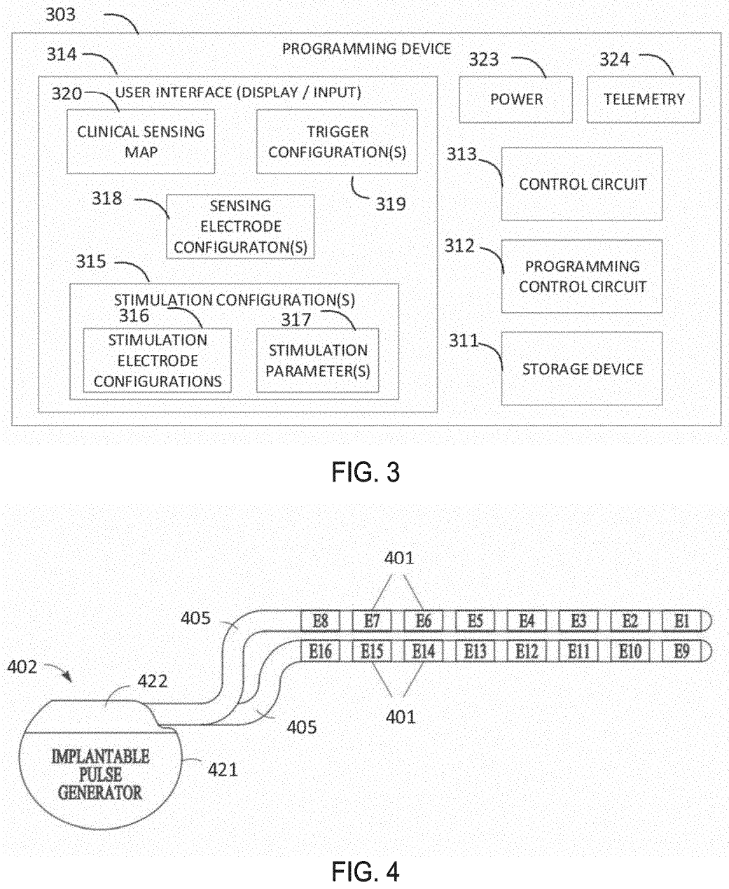

FIG. 3 illustrates, by way of example, an embodiment of a programming device 303, such as the programming device 103 illustrated in FIG. 1. The programming device may have a power source 323 and may have telemetry 324 for use to communicate with the implantable device. The programming device 303 may include a storage device 311, a programming control circuit 312, a control circuit 313 and a user interface 314. The storage device 311 may include instructions for evaluating sensing-capable electrodes which will be discussed in more detail below, impedance measurements performed during one or more evaluations such as may be used to record individual impedance measurements or detect trends in impedance measurements, identifiers for the sensing-capable electrodes, the status of the sensing-capable records, recorded violations, and the like. The storage device 311 may store neuromodulation parameter sets or programs, and/or a plurality of waveform building blocks to create the programs. The programming control circuit 312 may generate a plurality of stimulation parameters that control the delivery of the neuromodulation waveform. The control circuit 313 may receive a signal and may adjust the values of the plurality of stimulation parameters based on the received signal. The received signal may include information about a position of an electrode relative to the patient. The control circuit 313 may determine at least one stimulation parameter based on the position of the electrode relative to the patient.

In an example, the user interface 314 may include, but is not limited to, a touchscreen. In an example, the user interface may include any type of presentation device, such as interactive or non-interactive screens, and any type of user input devices that allow the user to edit the waveforms or building blocks and schedule the programs, such as touchscreen, keyboard, keypad, touchpad, trackball, joystick, and mouse. In an example, the circuits of neuromodulation system, including its various embodiments discussed in this document, may be implemented using a combination of hardware and software. For example, the circuit of the user interface, the stimulation control circuit, and the programming control circuit, including their various embodiments discussed in this document, may be implemented using an application-specific circuit constructed to perform one or more particular functions or a general-purpose circuit programmed to perform such function(s). Such a general-purpose circuit may include, but is not limited to, a microprocessor or a portion thereof, a microcontroller or portions thereof, and a programmable logic circuit or a portion thereof. The user interface 314, which may be an embodiment of user interface 104 in FIG. 1, may provide a display and user-inputs for use by the user to create stimulation configurations 315 including the stimulation electrode configuration(s) 316 and stimulation parameter(s) 317. Stimulation electrode configuration(s) 316 represent the active electrodes that are used to deliver neurostimulation to tissue. The stimulation parameter(s) 317 may represent the parameters of the energy delivered to the active electrodes. Stimulation parameter(s) 317 may include fractionalization information to control the distribution of energy across the active electrodes by individually controlling the energy (e.g. current) delivered to each individual active electrode. The user interface 314 may be configured for use to create sensing electrode configuration(s) 318, to create or adjust trigger configurations 319 for setting condition(s) that control when the evaluation of sensing-capable electrodes is triggered, and view and/or modify reports concerning the sensing-capable electrodes such as a clinical sensing map 320.

FIG. 4 illustrates, by way of example and not limitation, an embodiment of an implantable pulse generator (IPG) 402 and percutaneous leads 405. One of the neuromodulation leads may have eight electrodes (labeled E1-E8), and the other neuromodulation lead may have eight electrodes 401 (labeled E9-E16). The actual number and shape of leads and electrodes may, of course, vary according to the intended application. The IPG may comprise an outer case 421 for housing the electronic and other components (described in further detail below), and a connector 422 to which the proximal ends of the neuromodulation leads mates in a manner that electrically couples the electrodes to the electronics within the outer case. The outer case may be composed of an electrically conductive, biocompatible material, such as titanium, and forms a hermetically sealed compartment wherein the internal electronics are protected from the body tissue and fluids. In some examples, the outer case may serve as an electrode.

The IPG 402 may be configured to sense ECAPs using at least some of the electrodes. As will be discussed in further detail below, the IGP may be configured to control which of the electrodes are available to be active for sensing, which of the electrodes are active for sensing, and the sensing configuration (e.g., signal or differential pair) of the active electrode. By way of example and not limitation, electrodes E1, E2, E3 E9, E10 and E11 may be identified as available sensing-capable electrodes, and electrodes E1 and E2 may be activated for sensing as a differential pair, and electrodes E3, E9, E10 and E11 remain inactive for sensing. The electrodes that are identified as available sensing-capable electrodes may be specific to a particular implant, as it may depend on the location of the electrodes with respect to the nerve traffic being sensed and may depend on whether other electrodes are being used to deliver a therapy. The LPG is also configured to perform the evaluation of sensing-capable electrodes for use in selecting and maintaining the active electrodes for sensing ECAPs.

In an example, the IPG 402 includes a battery and pulse generation circuitry that delivers the electrical modulation energy in the form of one or more electrical pulse trains to the electrode array in accordance with a set of modulation parameters programmed into the IPG. Such modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters that may define the pulse amplitude (which may be measured in milliamps or volts depending on whether the IPG supplies constant current or constant voltage to the electrode array), pulse duration (which may be measured in microseconds), pulse rate (which may be measured in pulses per second), and burst rate (which may be measured as the modulation on duration X and modulation off duration Y).

In an example, electrical modulation may occur between two (or more) activated electrodes, one of which may be the IPG case. Modulation energy may be transmitted to the tissue in a monopolar or multipolar (e.g., bipolar, tripolar, etc.) fashion. Monopolar modulation may occur when a selected one of the lead electrodes is activated along with the case of the IPG, so that modulation energy is transmitted between the selected electrode and case. Bipolar modulation may occur when two of the lead electrodes are activated as anode and cathode, so that modulation energy is transmitted between the selected electrodes. For example, electrode E3 on the first lead may be activated as an anode at the same time that electrode E11 on the second lead is activated as a cathode. Tripolar modulation may occur when three of the lead electrodes are activated, two as anodes and the remaining one as a cathode, or two as cathodes and the remaining one as an anode. For example, electrodes E4 and E5 on the first lead may be activated as anodes at the same time that electrode E12 on the second lead is activated as a cathode. The modulation energy may be delivered between a specified group of electrodes as monophasic electrical energy or multiphasic electrical energy.

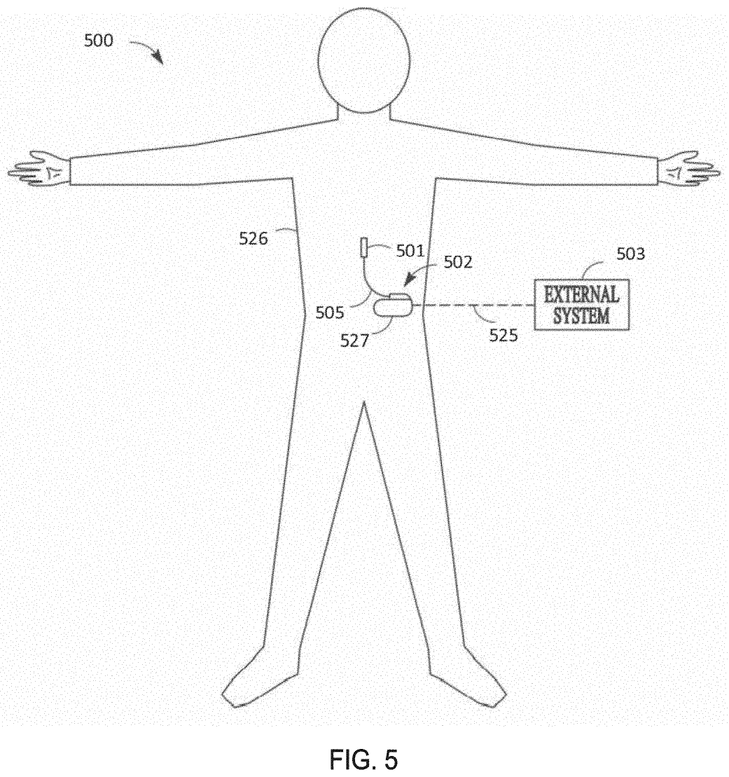

FIG. 5 illustrates an example of an implantable neuromodulation system and portions of an environment in which system may be used. The system 500 may include an implantable system 502, an external system 503, and a telemetry link 525 providing for wireless communication between the implantable system 502 and the external system 503. The implantable system 502 is illustrated in FIG. 5 as being implanted in the patient's body 526. By way of example and not limitation, the implanatable system may be configured for use to deliver spinal cord stimulation and/or sense nerve traffic in or near the spinal cord. The system may be configured to sense electrical activity in other tissue of interest (e.g., brain activity, activity in peripheral nerves such as, but not limited to, the vagus nerve, or muscles).

The implantable system may include an implantable stimulator 527 (also referred to as an implantable pulse generator, or IPG), a lead system 505, and electrodes 501, which may represent an embodiment of stimulation device, lead system, and electrodes, respectively. The external system 503 may represent an embodiment of programming device. In an example, the external system includes one or more external (non-implantable) devices each allowing the user and/or the patient to communicate with implantable system. In an example, the external system may include a programming device intended for the user to initialize and adjust settings for the implantable stimulator and a remote control device intended for use by the patient. For example, the remote control device may allow the patient to turn the implantable stimulator on and off and/or adjust certain patient-programmable parameters of the plurality of stimulation parameters.

The sizes and shapes of the elements of the implantable system and their location in the body are illustrated by way of example and not by way of restriction. In various examples, the present subject matter may be applied in any implantable or external device configured to sense electrical activity, such as nerve activity (i.e., ECAPs), within a patient.

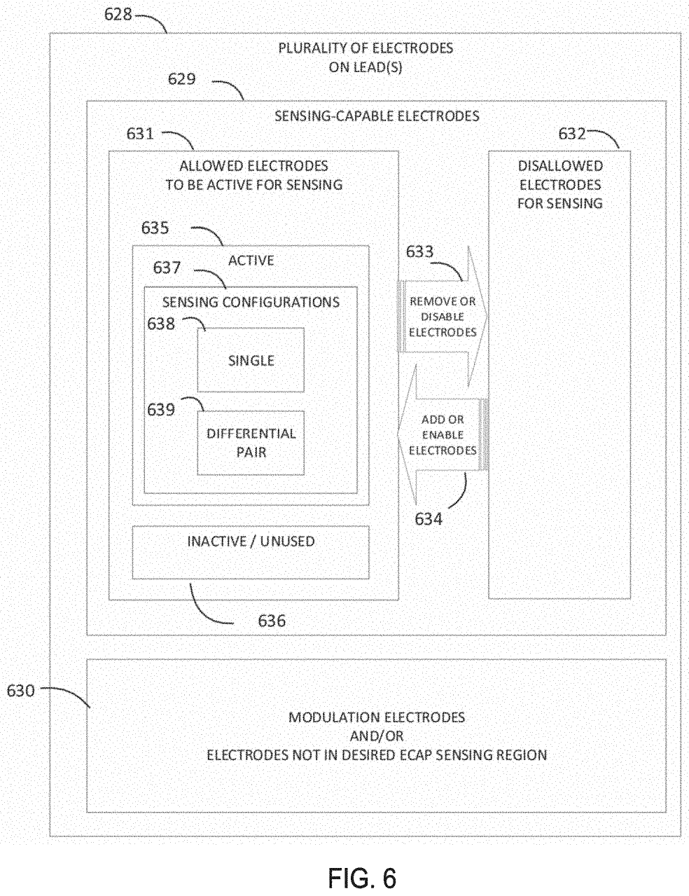

FIG. 6 provides, by way of example and not limitation, an illustration of sensing-capable electrodes for a lead, enabled (or allowed) electrodes to be active for sensing, and active ones of the electrodes for sensing. The electrodes in the system may include a plurality of electrodes on one or more leads, and may also include electrode(s) on the housing (e.g., can electrode(s)) of the implantable device. It is noted that lead(s) are used as an embodiment, but that the present subject matter may be implemented with leadless electrode(s) configured to wirelessly communicate with each other and/or with the implantable device.

A plurality of electrodes 628 may be on one or more leads. Some of these electrodes may be selected or otherwise identified to be sensing-capable electrodes 629, and some of these electrodes may not be considered to be a sensing-capable electrode for reasons such as, but not limited to, the electrode position being too far away from the neural tissue of interest. Some of these electrodes not considered to be a sensing-capable electrode may be used for other purposes (e.g., modulation electrodes 630).

The present subject matter may evaluate the sensing-capable electrodes 629 to determine which electrodes are allowed to be active for sensing 631 and which electrodes are disallowed for sensing 632. This evaluation may be used as at least a partial basis to reclassify or move, as illustrated at 633, previously-allowable electrodes 631 to the disallowed electrodes 632. This evaluation may be used as at least a partial basis to reclassify or move, as illustrated at 634, previously disallowed (or disabled) electrodes 632 to allowed (or enabled) electrodes for sensing 631. Of the sensing-capable electrodes 629 that are allowed to be active 631, the system may be configured to select those electrodes that are active for sensing 635. The non-selected electrodes are inactive or unused 636. The system may be further configured to configure or identify the sensing configuration 637 for the active electrodes. Examples of such sensing configurations 637 for a given one of the active electrodes may include using the given electrode alone (e.g., single electrode sensing 638) or using the given electrode with another electrode to provide different pair sensing 639.

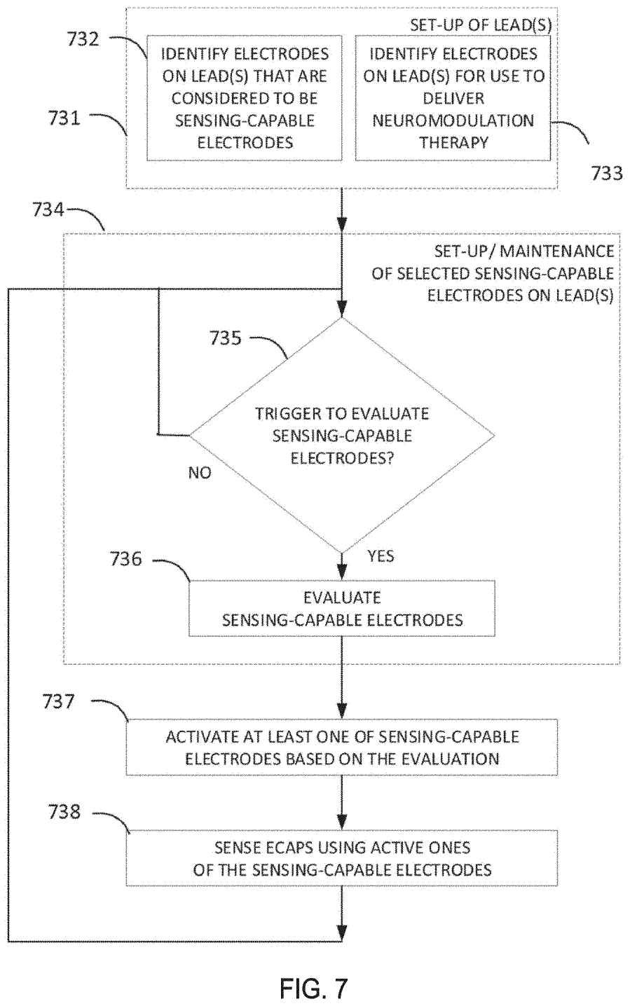

FIG. 7 illustrates, by way of example and not limitation, a method for configuring and using sensing electrodes to sense evoked compound action potentials (ECAPs). The illustrated method performs a process to set-up lead(s) 731. The process to set-up leads 731 may include a process to initially select or otherwise identify the electrode(s) on the lead(s) that are considered to be sensing-capable electrodes 732, which were illustrated at 629 in FIG. 6. The process to set-up leads 731 may also include identifying other electrode(s) on the lead(s) for use to deliver a neuromodulation therapy 732. The illustrated method may also perform a process 734 to set-up the electrodes that have been selected to be sensing-capable electrodes or to maintain previously set-up sensing-capable electrodes. The process 734 may include receiving a trigger signal 735 indicative of a trigger to evaluate sensing capabilities of the sensing capable electrodes. The trigger signal may be indicative of a user-input request to initiate the evaluation of the sensing capable electrodes. For example, the user may actuate a control element on a display screen to provide the evaluation command, or may press a button or provide a voice command to initiate the evaluation. By way of example and not limitation, the trigger may be based on a programmed evaluation schedule. For example, the evaluation schedule may trigger an evaluation of the sensing-capable electrodes at regular intervals (e.g. daily, weekly, monthly and the like) or at irregular intervals. For example, the programmed schedule may be use a shorter interval after implantation or after a neuromodulation program change and longer intervals as time progresses after the implantation or neuromodulation program change. The trigger may be provided every pulse in the delivered neuromodulation or after a certain count of pulses have been delivered. By way of another example and not limitation, the trigger may be indicative of one sensor output or may be indicative of more than one sensor output (e.g., blended sensor outputs). For example, various physiological sensor outputs may, individually or as a blended sensor, provide an indication of a response to a therapy (e.g., pain or lack thereof). If the sensor output(s) do not correlate well with what is expected based on the measured ECAPs, then the system may create a trigger to evaluate the sensing-capable electrodes. In another example, the sensor output(s) may simply indicate a trigger for which there is a significant likelihood of lead migration such that it may be desirable to reevaluate the sensing-capable electrodes (e.g., detected significant patient activity or motion, or posture changes). If there is a trigger at 735, then the process will evaluate the sensing capable electrodes 736 to determine which of the sensing-capable electrodes are allowed to be active as illustrated at 631 in FIG. 6 and the sensing-capable electrodes that are disabled or not allowed to be active as illustrated at 632 in FIG. 6. At 737, at least one of the sensing-capable electrodes may be activated based, at least in part, on the evaluation (i.e., based, at least in part, on the electrodes that are enabled or allowed to be active to sense ECAPs). At 738, active ones of the sensing-capable electrodes may be used to sense ECAPs.

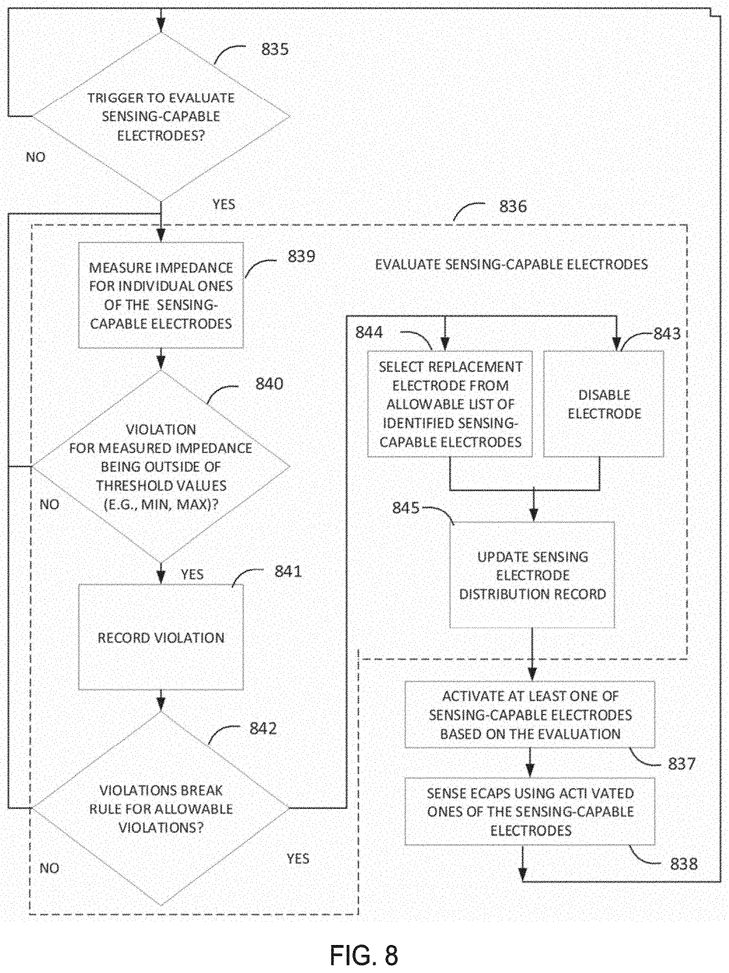

FIG. 8 illustrates, by way of example and not limitation, a method for evaluating sensing-capable electrodes 836 in response to receiving a trigger 835. The evaluation illustrated at 836 may be a more specific example for the evaluation illustrated at 736 in FIG. 7. The process for evaluating sensing-capable electrodes 836 may including measuring impedance for individual ones of the sensing-capable electrodes 839. Some embodiments may record all measured impedances over a window of time (e.g., a rolling window of time corresponding to a period of interest (e.g., hour(s), day(s), week(s), month(s)) to allow reporting of these impedances. Thus, the trends of the impedances for individual electrodes may be analyzed. Also, the trends of impedances for an electrode may be compared to the trends of impedances for other electrodes in the pool of allowable sensing-capable electrodes. These reporting data may be presented to a user on an external device. At 840, it is determined whether a violation occurred attributed to the measured impedances between outside of window of acceptable values (e.g., outside of minimum and maximum threshold values for the impedances). The window of acceptable values may be determined for a specific patient implanted with the lead(s) in a clinical setting. If there is no violation, the process returns to measure the impedance for the next electrode (until all electrode impedances of concern have been measured). If there is a violation, the system records the violation at 841. Some embodiments may record all violations over a window of time (e.g., a rolling window of time corresponding to a period of interest (e.g. hour(s), day(s), week(s), month(s)) to allow reporting of these violations. These violations may be presented to a user on an external device. At 842, the system may check to see if the current violation (and any previously-recorded violation(s)) break a rule for allowable impedance violations. If the violation(s) do not break a rule, then the process may return to measure the next electrode impedance at 839 (until all electrode impedances of concern have been measured). If the violation(s) break a rule at 842, then that electrode may be disabled or disallowed 843 from use to sense ECAPs. Also, a replacement electrode may be selected from the allowable sensing-capable electrodes 844. A sensing electrode distribution record may be updated at 845. The record may track the sensing-capable electrodes that are allowed to be active, the active ones of the sensing-capable electrodes, and the sensing configuration of the active one of the sensing-capable electrodes. The record may also track when the evaluation occurred and when a status of an electrode changes (e.g., a previously-enabled electrode becomes disabled for use in sensing ECAPs or a previously-disabled electrode becomes enabled for use in sensing ECAPs). Similar to FIG. 7 at 737, the at least one of the sensing-capable electrodes may be activated based, at least in part, on the evaluation 837 (i.e., based, at least in part, on the electrodes that are enabled or allowed to be active to sense ECAPs); and similar to FIG. 8 at 738, active ones of the sensing-capable electrodes may be used to sense ECAPs 838.

The rule(s) for allowable violations 842 may be designed to prevent excessive toggling of a sensing-capable electrode between an allowed status and a disallowed states. Thus, for example, the rules may require predetermined number of violations without intervening impedance measurements that do not cause a violation. Another example of a rule may require a total number of measurements, and further require that a certain percentage of those measurements are violations before proceeding to disabling the electrode for use to sense. Another example of a rule may track how often an electrode moves in and out of being acceptable. If an electrode moves back and forth from being acceptable a certain number of times, over a certain period, then it may be removed from the sensing capable electrode pool.

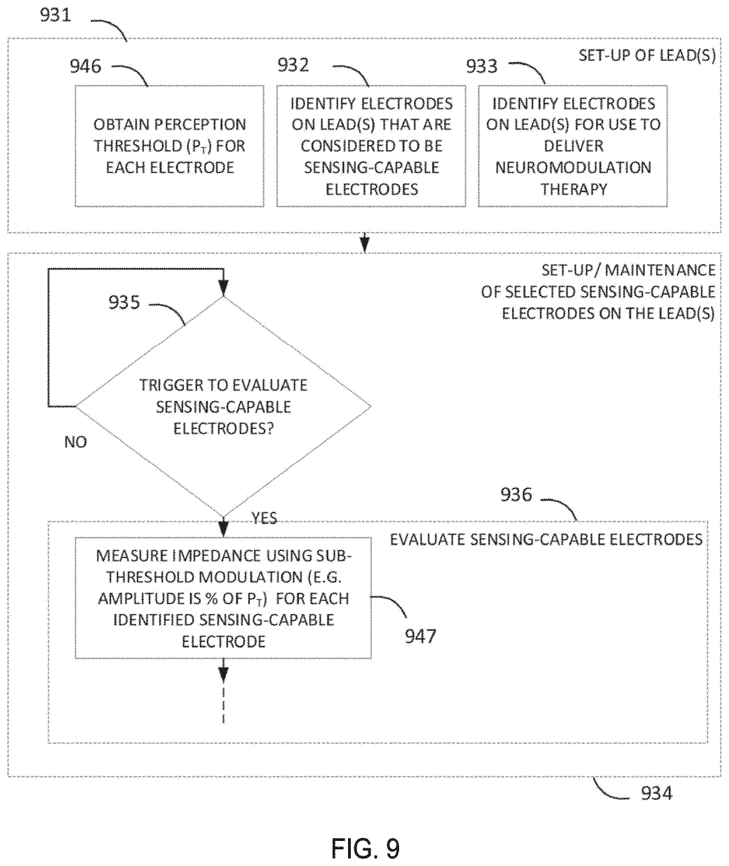

FIG. 9 illustrates, by way of example and not limitation, a method for configuring and using sensing electrodes to sense evoked compound action potentials (ECAPs) where the impedance is measured using sub-threshold energy. It may be desirable to use sub-threshold energy to measure electrode impedance so that the patient does not feel anything when the impedance is being sensed for each sensing-capable electrode. Various parameters (amplitude, pulse width, frequency) may be adjusted such that the energy is sub-threshold. The method in FIG. 9 controls the amplitude for the delivered energy so that it is below previous-determined perception thresholds for each electrode. However, other parameters may be controlled to provide the sub-threshold energy. FIG. 9 has similarities to FIG. 7, as it illustrates the set-up of leads 931 and a process 934 to set-up the electrodes that have been selected to be sensing-capable electrodes or to maintain previously set-up sensing-capable electrodes. The process to set-up leads 931 may include a process to initially select or otherwise identify the electrode(s) on the lead(s) that are considered to be sensing-capable electrodes 932, and may also include identifying other electrode(s) 933 on the lead(s) for use to deliver a neuromodulation therapy. Part of the process for setting up leads after implantation may be to measure the perception threshold (P_{T}) of each electrode. The electrode-specific P_{T} may be used to normalize the energy delivery to each individual electrode, as it accounts for electrode/tissue coupling for specific ones of the electrodes by determining how the patient perceives energy delivered from each electrode. Such a normalization process provide better ability to deliver the intended energy into the tissue. P_{T} measurements generally rely on the patient's subject determination of when they feel delivery of the stimulation (e.g., feel paresthesia). Some embodiments may use physiological sensors in an effort to provide a more objective determination of the electrode-tissue interface. Thus, a certain “level” of energy will provide a similar patient response. These measurements 946 may be used to determine the appropriate energy used to obtain the impedance measurements at 947. For example, after a trigger is received at 935, the impedance of each identified sensing-capable electrode may be measured using sub-threshold energy 947. This sub-threshold energy may be determined as a percentage of P_{T} (e.g., 50% to 90% of P_{T} or 60% to 80% of P_{T}). This sub-threshold energy may be determined as an offset below the P_{T} (e.g., “x” mA below P_{T}).

The illustrated method may also perform a process 734 to set-up the electrodes that have been selected to be sensing-capable electrodes or to maintain previously set-up sensing-capable electrodes. The process 734 may include receiving a trigger signal 735 indicative of a trigger to evaluate sensing capabilities of the sensing capable electrodes.

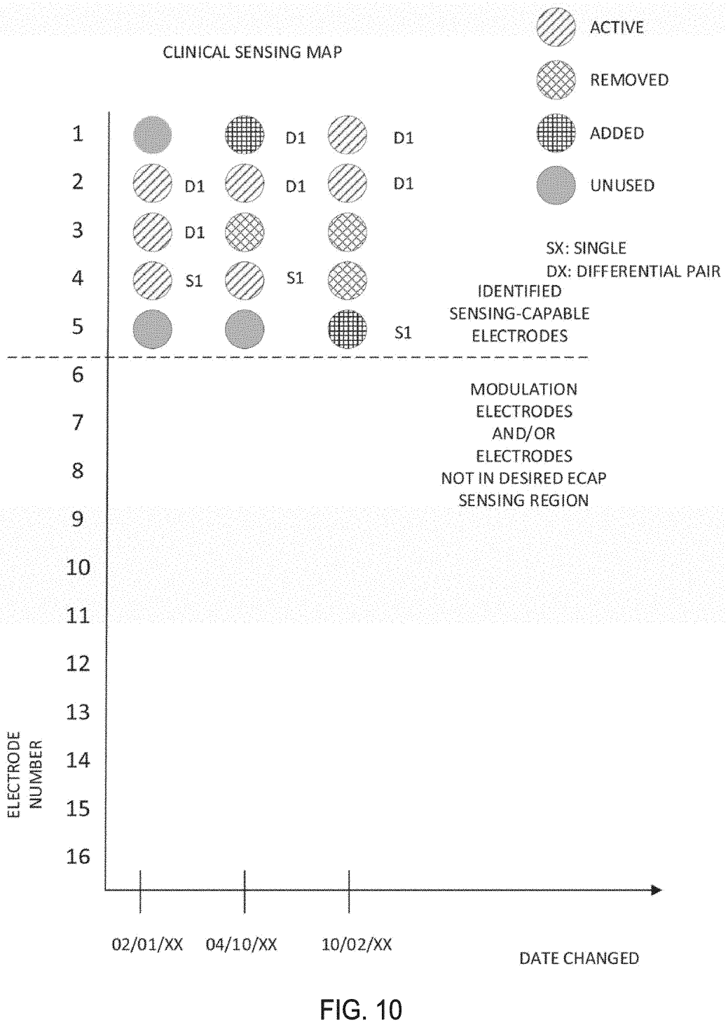

FIG. 10 illustrates, by way of example and not limitation, an embodiment of reporting data provided in a form of a clinical sensing map. The clinical sensing map may be displayed on the user interface. By way of example and not limitation, the map may include a graph with vertical and horizontal axes. Electrode identifiers, such as electrode numbers, may be provided along the vertical axis. In the illustrated embodiment, the map corresponds to a system with 16 electrodes. The first five electrodes may be identified or otherwise characterized as sensing-capable electrodes. The remainder of the electrodes are not considered to be sensing-capable electrodes, as they may be used to deliver neuromodulation or may not be in an appropriate position to sense ECAPs. The horizontal axis may include time identifiers (e.g. dates), which may correspond to dates when the sensing-capable electrodes were evaluated. A representation of the sensing-capable electrodes may be provided, along with a status identifier for each of the sensing-capable electrodes at the time of the evaluation date. For example, the status identifiers may indicate the unused electrodes, the active electrodes, the removed or disabled electrodes, and the added or enabled electrodes. A removed or disabled electrode may be a type of unused electrode, and an added or enabled electrode may be a type of an active electrode. These status identifiers may be communicated by applying different colors and/or patterns to the electrodes, or may be communicated by labels. The map may also identify a sensing configuration for the active electrodes. For example, single sensing electrodes may be labeled with “S#” such as S1, S2, etc. and each of two differential pair electrodes may be labeled with “D#” such as D1. The report data used to generate the clinical sensing map may be provided using other report forms. For example, report data may be recorded in a matrix of per-electrode or per electrode group (differential for example) sensing, electrode identifiers, differential pair group numbers, number of violations, and average impedance measurement value for successive violations.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
receiving a trigger signal indicative of a trigger to evaluate sensing capabilities of the sensing capable electrodes;
responding to the received trigger signal by evaluating the sensing capabilities of the sensing-capable electrodes to assess or reassess which of the sensing-capable electrodes are available to be activated for sensing evoked compound action potentials (ECAPs);
activating at least one of the sensing-capable electrodes that are available to be activated based on the evaluating the sensing capabilities; and
sensing the ECAPs using the activated ones of the sensing-capable electrodes.

2. The method of claim 1, wherein the evaluating the sensing capabilities of the sensing-capable electrodes includes measuring an impedance corresponding to each of at least one of the sensing-capable electrodes, and evaluating the measured impedance against threshold values to assess or reassess which of the sensing-capable electrodes are available to be activated for sensing ECAPs.

3. The method of claim 2, further comprising disabling at least one electrode, based on the evaluating the measured impedance against the threshold values, from being available to be activated.

4. The method of claim 2, further comprising enabling at least one electrode, based on the evaluating the measured impedance against the threshold values, to be available to be activated.

5. The method of claim 1, further comprising receiving a user-input via a user input, wherein the trigger signal is indicative of the user input.

6. The method of claim 1, further comprising accessing a scheduled programmed in a memory, wherein the trigger signal is provided in accordance with the programmed schedule.

7. The method of claim 1, further comprising using at least one sensor to sense at least one physiological parameter and providing the trigger signal based on the sensed at least one physiological parameter.

8. The method of claim 1, further comprising monitoring the sensed ECAPs and providing the trigger signal based on the monitored sensed ECAPs.

9. The method of claim 1, further comprising reconfiguring sensing configurations to create a different differential pair when at least one electrode in an existing differential pair is to be removed.

10. The method of claim 1, further comprising reconfiguring sensing configurations to automatically replace a single electrode when another single electrode is removed.

11. The method of claim 1, further comprising generating a sensing map report that identifies at least one electrode added to the sensing-capable electrodes that are available to be activated for sensing ECAPs.

12. The method of claim 1, further comprising generating a sensing map report that identifies at least one electrode removed from the sensing-capable electrodes that are available to be activated for sensing ECAPs.

13. The method of claim 1, further comprising generating a sensing map report that identifies the sensing-capable electrodes that are available to be activated for sensing ECAPs.

14. The method of claim 1, wherein the evaluating the sensing capabilities of the sensing-capable electrodes includes measuring impedance for individual ones of the sensing capable electrodes.

15. The method of claim 1, wherein the evaluating the sensing capabilities includes comparing a measured impedance correspond to an electrode to threshold values for the electrode.

16. The method of claim 15, wherein the evaluating the sensing capabilities further includes recording a violation when the measured impedance is outside of the threshold values, determining that recorded violations break a rule for allowable violations, and updating the sensing-capable electrodes that are available to be activated for sensing ECAPs.

17. The method of claim 16, wherein the evaluating the sensing capabilities further includes updating a sensing electrode distribution record.

18. A non-transitory machine-readable medium including instructions, which when executed by a machine, cause the machine to perform a method comprising:
receiving a trigger signal indicative of a trigger to evaluate sensing capabilities of the sensing capable electrodes;
responding to the received trigger signal by evaluating the sensing capabilities of the sensing-capable electrodes to assess or reassess which of the sensing-capable electrodes are available to be activated for sensing evoked compound action potentials (ECAPs);
activating at least one of the sensing-capable electrodes that are available to be activated based on the evaluating the sensing capabilities; and
sensing the ECAPs using the activated ones of the sensing-capable electrodes.

19. A system, comprising:
an implantable device, including sensing-capable electrodes;
a controller configured to:
receive a trigger signal indicative of a trigger to evaluate sensing capabilities of the sensing capable electrodes;
respond to the received trigger signal by evaluating the sensing capabilities of the sensing-capable electrodes to assess or reassess which of the sensing-capable electrodes are available to be activated for sensing evoked compound action potentials (ECAPs);
activate at least one of the sensing-capable electrodes that are available to be activated based on the evaluating the sensing capabilities; and
sense the ECAPs using the activated ones of the sensing-capable electrodes.

20. The system of claim 19, wherein the controller is configured to respond to the received trigger by measuring an impedance corresponding to each of at least one of the sensing-capable electrodes, and evaluating the measured impedance against threshold values to assess or reassess which of the sensing-capable electrodes are available to be activated for sensing ECAPs.
